(19) United States
(12) Patent Application Publication
(10) Pub. No.: US 2026/0338783 A1
(43) Pub. Date: Oct. 29, 2026

(54) TUMOR RESIDENT PROTEIN MARKER PANEL FOR PREDICTING RECURRENCE IN BILE DUCT CANCER PATIENTS

(71) Applicant: ALBERT-LUDWIGS-UNIVERSITÄT FREIBURG KÖRPERSCHAFT DES ÖFFENTLICHEN RECHTS, Freiburg (DE)

(72) Inventors: Oliver SCHILLING, Basel (CH); Peter BRONSERT, Vörstetten (DE); Tilman WERNER, Freiburg (DE); Klara BUDAU, Freiburg (DE); Niko PINTER, Kappelrodeck (DE); Miguel DO JÉSUS COSENZA CONTRERAS, Freiburg (DE)

(21) Appl. No.: 19/485,457

(22) PCT Filed: Apr. 9, 2024
(86) PCT No.: PCT/EP2024/059595

§ 371 Date: Nov. 18, 2025

(30) Foreign Application Priority Data
May 22, 2023 (DE) 10 2023 113 350.0

(51) Int. Cl. G01N 33/575

(52) U.S. Cl. CPC G01N 33/57557; G01N 2333/4722; G01N 2333/78; G01N 2333/904; G01N 2333/988

(57) ABSTRACT

The present invention relates to a method for determining the risk for recurrence in a bile duct cancer (CCA) patient, comprising detecting the expression levels of a set of relevant markers. Based on the risk as determined, the invention allows for providing a prognosis for a bile duct cancer (CCA) patient, monitoring the status of bile duct cancer (CCA) in a patient, and to identify a bile duct cancer (CCA) patient in need of adjuvant therapy. The present invention also relates to an array of improved markers for determining the risk for recurrence in a bile duct cancer (CCA) patient.

The present invention relates to a method for determining the risk for recurrence in a bile duct cancer (CCA) patient, comprising detecting the expression levels of a set of relevant markers. Based on the risk as determined, the invention allows for providing a prognosis for a bile duct cancer (CCA) patient, monitoring the status of bile duct cancer (CCA) in a patient, and to identify a bile duct cancer (CCA) patient in need of adjuvant therapy. The present invention also relates to an array of improved markers for determining the risk for recurrence in a bile duct cancer (CCA) patient.

### BACKGROUND OF THE INVENTION

Bile duct cancers—also termed cholangiocarcinoma or CCA—comprise the second most prevalent class of hepatic tumors, with 15% of all liver malignancies falling into this group. In the United States, there are 5,000 new cases of biliary cancer per year. In Korea, gall bladder and biliary cancer accounts for 3.6% of all cancers. According to their anatomical location, CCA can be categorized into three subtypes: intrahepatic (iCCA), perihilar or distal adenocarcinomas. Although CAA subtypes possess different genetic, histologic, and clinical features, they all are characterized by a late onset of clinical symptoms and usually diagnosed in advanced tumor stages. iCCA tumors are largely inaccessible to histological probing and are so far only insufficiently described, despite a rising incidence worldwide. Due to a lack of standardized diagnostic imaging criteria or reliable tumor markers, diagnostics and personalized treatment of iCCA are particularly difficult. Furthermore, patients frequently develop local recurrences.

iCCAs derive from small ducts and ductules inside the liver and remain symptom-free in early stages. After progressed growth, iCCAs are usually diagnosed by unspecific B symptoms comprising jaundice, fatigue, and loss of weight. Risk factors are poorly defined, and most cases occur without presence of a known risk. Nevertheless, inflammatory and fibrosis promoting events in the bile duct such as stasis, parasitic or viral infections, alcohol and tobacco abuse or obesity and diabetes seem to advance the formation of iCCA.

As the majority of iCCA patients initially presents with tumor sizes larger than 6 cm, surgical tumor resection, including hemi-hepatectomies, remains the first-line treatment option with, however, only slight chances for recovery. After surgery, pathological assessment remains complicated since there is no internationally agreed upon staging system for iCCA. The American Joint Committee on Cancer (AJCC) TNM system is most used for tumor grading but lacks in predictive power due to its coarse classification criteria and an exclusion of potential predictive factors. Despite the high number of patients with tumor recurrences, applying individual prognoses and tailored therapy regimens therefore remains challenging.

WO2010100862A1 discloses a method for detecting and determining intrahepatic cholangiocarcinoma, which is a malignant tumor primarily occurring in the liver, in the early stage in a simple manner with high sensitivity and high reliability. Also disclosed is a kit for achieving the method. In the method, intrahepatic cholangiocarcinoma can be detected by detecting a cancer marker in a sample, wherein the cancer marker is a sugar chain biomarker comprising a lectin WFA (wisteria fioribunda agglutinin)-binding glycoprotein derived from intrahepatic cholangiocarcinoma. The intrahepatic cholangiocarcinoma detection method enables the clear distinction of intrahepatic cholangiocarcinoma from hepatocellular carcinoma, and also enables the detection and determination of intrahepatic cholangiocarcinoma in the early stage with clinically acceptable applicability, sensitivity and accuracy.

WO2018020530A1 addresses the problem of providing a method for collecting highly accurate diagnostic data and a diagnostic kit, both of which are useful for the diagnosis of the occurrence of extrahepatic bile duct cancer, intrahepatic bile duct cancer or gallbladder cancer. The concentration of CEACAM1 in a blood sample collected from a subject is detected using a diagnostic kit including an antibody capable of binding specifically to CEACAM1 or a labeled product of the antibody or the like, whereby it becomes possible to obtain data from which it is diagnosed that a subject is highly probably suffering from extrahepatic bile duct cancer, intrahepatic bile duct cancer or gallbladder cancer when the concentration of CEACAM1 is higher than that in a blood sample from a normal person or is higher than a given threshold value (cut-off value).

JP2008072952A provide a marker gene useful in differential diagnosis of intrahepatic cholangiocarcinoma (ICC) and a method of differential diagnosis of the intrahepatic cholangiocarcinoma (ICC) using the marker gene. The reagent for detecting intrahepatic cholangiocarcinoma contains a nucleotide comprising a base sequence of one gene or at least two genes selected from among a group comprising four kinds of genes: (1) insulin-like growth factor-binding protein 5 (IGFBP5), (2) Claudin4 (CLDN4), (3) PDZ and LIM domain 7 (PDLIM7), and (4) Biglycan (BGN), excluding Claudin4 or a nucleotide that contains a partial sequence thereof.

KR 20170105184A discloses a method based on the difference in the expression of 794 cholangiolary differentiation signatures (CD signature) of intrahepatic cholangiocarcinoma patients showing bad prognosis and intrahepatic cholangiocarcinoma patients showing improved prognosis, and according to the present invention, it was confirmed that the CD signature not only shows the expression difference between ICC subtypes, and also can diagnose the prognostic subtypes of ICC more specifically than the cholangiolar pancreatic duct differentiation signature (CP signature), and thus the gene or protein encoded by the gene can be used as a biomarker for predicting prognosis of intrahepatic cholangiocarcinoma in independent or collaborative manners. One of the markers as described is VTN.

Despite the above progress, there is still a need to provide novel diagnostic tools are urgently needed to assess tumor aggressiveness and predict each patient's therapeutic needs directly after surgery.

It is therefore an object of the present invention to provide these diagnostic tools, and to develop improved respective anti-tumor therapies and strategies. Other objects and advantages of the present invention will become apparent to the person of skill when studying the following more detailed description of the present invention, including the Figures and examples.

According to a first aspect of the present invention the above object is solved by providing a method for determining the risk for recurrence in a bile duct cancer (CCA) patient, comprising: obtaining a tissue sample from the patient, and detecting the expression levels of at least one marker selected from Vitronectin (VTNC), biglycan (BGN), histone 10 (H10), L-lactate dehydrogenase A chain (LDHA), fructose-bisphosphate aldolase A (ALDOA), and SKI oncoprotein (SKI) in the sample, wherein, when compared to a control sample, an increase of the expression level as detected for at least one marker selected from VTNC, BGN, and H10 is indicative for a low risk for recurrence, and a decrease of the expression level as detected for at least one marker selected from LDHA, ALDOA, and SKI is indicative for a high risk for recurrence.

The inventors identified two proteomic clusters in iCCA patients, which exhibit distinct biological motifs and different prognoses regarding their time to relapse (TTR). In a second, independent analysis via CPHM, the present invention identified a set of 130 proteins, whose expression patterns correlate best with patient's TTR. The inventors confirmed the detected proteins to be also differentially regulated among the clusters, which indicates their expression levels to be representative for the activity of biological processes determing the TTR. After further analysis, the inventors identified a set of six marker proteins to predict iCCA patient's TTR, preferably shortly after initial surgery and tumor resection.

Preferred is the method for determining the risk for recurrence in a bile duct cancer (CCA) patient according to the present invention, wherein the least one marker is selected from the group consisting of VTNC, ALDO, and a combination of all six markers.

Further preferred is the method for determining the risk for recurrence in a bile duct cancer (CCA) patient according to the present invention, wherein the tissue sample is selected from a tumor sample from an intrahepatic (iCCA), perihilar and distal adenocarcinoma, preferably taken at the time of initial surgery.

Even further preferred is the method for determining the risk for recurrence in a bile duct cancer (CCA) patient according to the present invention, wherein said method is performed as a first line-therapy testing all six markers to determine the risk of an iCCA patient shortly after initial surgery.

According to the present invention, any method suitable to detect the expression of the markers may be used, as is known to the person of skill. Preferred is a method according to the present invention, wherein the detection of the expression of the at least one marker in the sample comprises a method selected from at least one of ELISA, immunohistochemical (IHC) staining, proteolysis, bicinchoninic acid assay, and mass spectrometry, preferably comprising a label-free quantitation (LFQ).

In the context of the present invention, a “control sample” can be any sample that is suitable for a comparison with the sample forming the basis for the method according to the present invention. Control samples can be samples obtained or derived from healthy patients or groups of patients, samples as taken earlier from the same patient or taken from patient or treatment groups. The control samples include/comprise at least one marker of the present invention.

In the context of the present invention, the term “marker” or “biomarker” shall mean a biological molecule, such as a protein or mRNA or detectable fragment thereof, related to the proteins VTNC, BGN, H10, LDHA, ALDOA, and SKI as defined herein, that can be detected in a sample in order to determine the expression level of the at least one marker. The markers of the present invention may be in solution or attached to a solid carrier, such as a bead or array. The marker may be suitably labelled, e.g., with an enzymatic, size or weight, antigenic or otherwise detectable label.

In the context of the present invention, the term “vitronectin” shall include both the mammalian, preferably human, protein or a functional fragment thereof, i.e., shall also include stretches and/or regions of the vitronectin polypeptide that can be detected. The term also includes the mRNA encoding the protein or a functional fragment thereof. The sequence of human vitronectin (VTNC_HUMAN) can be found in the UniProt database, accession number P04004. Vitronectin is a cell adhesion and spreading factor found in serum and tissues. Vitronectin interacts with glycosaminoglycans and proteoglycans, and is recognized by certain members of the integrin family and serves as a cell-to-substrate adhesion molecule. Inhibitor of the membrane-damaging effect of the terminal cytolytic complement pathway.

In the context of the present invention, the term “biglycan” shall include both the mammalian, preferably human, protein or a functional fragment thereof, i.e., shall also include stretches and/or regions of the biglycan polypeptide that can be detected. The term also includes the mRNA encoding the protein or a functional fragment thereof. The sequence of human biglycan (BGN) can be found in the UniProt database, accession number P21810. Biglycan may be involved in collagen fiber assembly.

In the context of the present invention, the term “histone 10” shall include both the mammalian, preferably human, protein or a functional fragment thereof, i.e., shall also include stretches and/or regions of the histone 10 polypeptide that can be detected. The term also includes the mRNA encoding the protein or a functional fragment thereof. The sequence of human histone 10 (H10) can be found in the UniProt database, accession number P07305. Histones H1 are necessary for the condensation of nucleosome chains into higher-order structures. The histones H1.0 are found in cells that are in terminal stages of differentiation or that have low rates of cell division.

In the context of the present invention, the term “L-lactate dehydrogenase A chain” shall include both the mammalian, preferably human, protein or a functional fragment thereof, i.e., shall also include stretches and/or regions of the L-lactate dehydrogenase A chain polypeptide that can be detected. The term also includes the mRNA encoding the protein or a functional fragment thereof. The sequence of human L-lactate dehydrogenase A chain (LDHA) can be found in the UniProt database, accession number P00338. LDHA interconverts simultaneously and stereospecifically pyruvate and lactate with concomitant interconversion of NADH and NAD+.

In the context of the present invention, the term “fructose-bisphosphate aldolase A” shall include both the mammalian, preferably human, protein or a functional fragment thereof, i.e., shall also include stretches and/or regions of the fructose-bisphosphate aldolase A polypeptide that can be detected. The term also includes the mRNA encoding the protein or a functional fragment thereof. The sequence of human fructose-bisphosphate aldolase A (ALDOA) can be found in the UniProt database, accession number P04075. In vertebrates, three forms of this ubiquitous glycolytic enzyme are found, aldolase A in muscle, aldolase B in liver and aldolase C in brain.

In the context of the present invention, the term “SKI oncoprotein” shall include both the mammalian, preferably human, protein or a functional fragment thereof, i.e., shall also include stretches and/or regions of the SKI oncoprotein polypeptide that can be detected. The term also includes the mRNA encoding the protein or a functional fragment thereof. The sequence of human SKI oncoprotein (SKI) can be found in the UniProt database, accession number P12755. May play a role in terminal differentiation of skeletal muscle cells but not in the determination of cells to the myogenic lineage. Functions as a repressor of TGF-beta signaling.

Preferred is the method for determining the risk for recurrence in a bile duct cancer (CCA) patient according to the present invention, the patient is selected from a cat, dog, mouse, rat, horse, sheep, goat, monkey, cow, or human, and is preferably selected from a human.

Another aspect of the present invention then relates to a method for providing a prognosis for a bile duct cancer (CCA) patient, comprising performing the method according to the present invention, and further comprising the step of providing a prognosis for the patient based on the risk for recurrence as determined, wherein, when compared to a control sample, an increase of the expression level as detected for at least one marker selected from VTNC, BGN, and H10 is indicative for a positive prognosis, and a decrease of the expression level as detected for at least one marker selected from LDHA, ALDOA, and SKI is indicative for a negative prognosis, wherein optionally the prognosis includes time-to-recurrence (TTR) and/or survival of the patient, as described herein.

Another aspect of the present invention then relates to a method for monitoring the status of bile duct cancer (CCA) in a patient, comprising performing the method according to the present invention, and further comprising the step of monitoring the status of bile duct cancer (CCA) in the patient based on comparing the results to results as obtained with an earlier sample obtained from the patient. In this embodiment, changes of the expression levels as detected for the at least one marker as disclosed are identified by comparing the results to results as obtained with an earlier sample obtained from the patient. These changes then provide the information as necessary for the attending physician, to start or amend a therapy for the patient (or a patient group) as disclosed herein.

Another aspect of the present invention therefore relates to a method for identifying a bile duct cancer (CCA) patient in need of adjuvant therapy, comprising performing the method according to the present invention, and further comprising the step of determining the patient as in need of adjuvant therapy, based on the risk for recurrence as determined. Similar to the above, changes of the expression levels as detected for the at least one marker as disclosed are identified by comparing the results to results as obtained with an earlier sample obtained from the patient. These changes then provide the information as necessary for the attending physician, to identify the patient (or a patient group) as disclosed herein, and to start or amend a therapy for the patient (or a patient group).

Preferred is a method according to the present invention, wherein the adjuvant therapy is selected from the group consisting of radiation therapy, chemotherapy, immunotherapy, hormone therapy, and targeted therapy. Preferred is a method according to the present invention, wherein the patient is undergoing an anti-cancer therapy, in particular a chemotherapy, surgery, radiation, or androgen ablation.

Another aspect of the present invention then relates to a method for identifying a compound that is active against a recurrence in a bile duct cancer (CCA) patient, comprising the steps of: a) providing at least one candidate compound with a subject suffering from CCA, and b) performing the method according to the present invention, wherein, when compared to the absence of the at least one candidate compound, an increase of the expression level as detected for at least one marker selected from VTNC, BGN, H10, LDHA, ALDOA, and SKI is indicative for a compound that is active against a recurrence.

In principle, any suitable candidate compound may be screened. Preferred is a method according to the present invention, wherein the candidate compound is selected from the group consisting of a chemical molecule, a molecule selected from a library of small organic molecules, a molecule selected from a combinatory library, an anti-cancer chemotherapeutic agent, a cell extract, in particular a plant cell extract, a small molecular drug, a protein, a protein fragment, a molecule selected from a peptide library, and an antibody or fragment thereof. Methods for screening are known in the art, preferred is a high-throughput method (HTS). Preferred is a screening in a suitable animal model.

Yet another aspect of the present invention then relates to a compound that is active against a recurrence in a bile duct cancer (CCA) patient as identified according to a method according to the present invention, or a pharmaceutical composition comprising said compound that is active against a recurrence in a bile duct cancer (CCA) patient, together with a pharmaceutically acceptable carrier. Preferred is a compound that is active against a recurrence in a bile duct cancer (CCA) patient according to the present invention, wherein the compound is selected from the group consisting of a chemical molecule, a molecule selected from a library of small organic molecules, a molecule selected from a combinatory library, a cell extract, in particular a plant cell extract, a small molecular drug, a protein, a protein fragment, a molecule selected from a peptide library, and an antibody or fragment thereof.

Another aspect of the present invention then relates to a method for producing a pharmaceutical composition that is active against a recurrence in a bile duct cancer (CCA) patient, comprising the steps of performing the method according to the present invention, and formulating the at least one compound as identified together with a pharmaceutically acceptable carrier.

Thus, in yet another aspect of the present invention, the selected or screened compound and/or compound for use as disclosed herein can be provided and/or is administered as a suitable pharmaceutical composition, such as a topical composition, tablet, capsule, granule, powder, sachet, reconstitutable powder, dry powder inhaler and/or chewable. Such solid formulations may comprise excipients and other ingredients in suitable amounts. Such solid formulations may contain e.g., cellulose, cellulose microcrystalline, polyvidone, in particular FB polyvidone, magnesium stearate and the like. The compound identified as outlined above, which may or may not have gone through additional rounds of modification, is admixed with suitable auxiliary substances and/or additives. Such substances comprise pharmacological acceptable substances, which increase the stability, solubility, biocompatibility, or biological half-life of the interacting compound or comprise substances or materials, which must be included for certain routes of application like, for example, intravenous solution, sprays, liposomes, ointments, skin crème, band-aids or pills.

It is to be understood that the present compound and/or a pharmaceutical composition comprising the present compound is for use to be administered to a human patient. The term “administering” means administration of a sole therapeutic agent or in combination with another therapeutic agent. It is thus envisaged that the pharmaceutical compositions of the present invention are employed in co-therapy approaches, i.e. in co-administration with another, or other medicaments or drugs and/or any other therapeutic agent which might be beneficial in the context of the methods of the present invention. Nevertheless, the other pharmaceutical composition of the present invention, medicaments or drugs and/or any other therapeutic agent can be administered separately from the compound as selected or screened and/or compound for use, if required, as long as they act in combination (i.e., directly and/or indirectly, preferably synergistically) with the present compound as selected or screened and/or for use.

Thus, the compounds as selected or screened and/or for use of the invention can be used alone or in combination with other active compounds—for example with medicaments already known for the treatment of bile duct cancer, whereby in the latter case a favorable additive, amplifying or preferably synergistically effect is noticed. Suitable amounts to be administered to humans range from 5 to 500 mg, in particular 10 mg to 100 mg. Of course, any dosage can be readily adjusted by the attending physician, if needed, based on, for example, other medical parameters of the patient to be treated.

Pharmaceutical compositions as used may optionally comprise a pharmaceutically acceptable carrier. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for respective compounds, for example topical, and will readily be able to choose suitable pharmaceutically acceptable carriers or excipients, depending, e.g., on the formulation and administration route of the pharmaceutical composition.

The therapeutics can be administered orally, e.g., in the form of pills, tablets, coated tablets, sugar coated tablets, hard and soft gelatin capsules, solutions, syrups, emulsions or suspensions or as aerosol mixtures. Administration, however, can also be carried out rectally, e.g., in the form of suppositories, or parenterally, e.g., in the form of injections or infusions, or percutaneously, e.g., in the form of ointments, creams or tinctures.

In addition to the aforementioned compounds as selected or screened and/or for use of the invention, the pharmaceutical composition can contain further customary, usually inert carrier materials or excipients. Thus, the pharmaceutical preparations can also contain additives, such as, for example, fillers, extenders, disintegrants, binders, glidants, wetting agents, stabilizers, emulsifiers, preservatives, sweetening agents, colorants, flavorings or aromatizers, buffer substances, and furthermore solvents or solubilizers or agents for achieving a depot effect, as well as salts for changing the osmotic pressure, coating agents or antioxidants. They can also contain the aforementioned salts of two or more compounds for use of the invention and also other therapeutically active substances as described above.

Another aspect of the present invention then relates to a method for treating or preventing recurrence in a bile duct cancer (CCA) patient, comprising performing a method according to the present invention, and administering an effective amount of the compound as identified or pharmaceutical composition or the pharmaceutical composition as produced to the patient, wherein the CCA is preferably selected from the group of intrahepatic (iCCA), perihilar and distal adenocarcinomas.

Another aspect of the present invention then relates to a compound that is active against a recurrence in a bile duct cancer (CCA) patient as identified according to the present invention, or the pharmaceutical composition as produced according to the present invention for use in the prevention or treatment of recurrence in a bile duct cancer (CCA) patient.

Yet another aspect of the present invention then relates to an array of markers for determining the risk for recurrence in a bile duct cancer (CCA) patient, comprising at least one marker selected from Vitronectin (VTNC), biglycan (BGN), histone 10 (H10), L-lactate dehydrogenase A chain (LDHA), fructose-bisphosphate aldolase A (ALDOA), and SKI oncoprotein (SKI). The array may be in solution or on a solid carrier, like beads or chips.

Another aspect of the present invention then relates to a kit for determining the risk for recurrence in a bile duct cancer (CCA) patient, comprising materials for performing the method according to the present invention, such as, for example, materials for label-free quantitation (LFQ), buffers, antibodies specifically binding to the markers, and/or the array of markers according to the present invention. Optionally, the kit contains additional materials, like a manual or the like. Preferred is the kit according to the present invention, which is suitable for high-throughput screening (HTS).

The present invention relates to the following items:

Item 1: A method for determining the risk for recurrence in a bile duct cancer (CCA) patient, comprising: obtaining a tissue sample from the patient, and detecting the expression levels of at least one marker selected from Vitronectin (VTNC), biglycan (BGN), histone 10 (H10), L-lactate dehydrogenase A chain (LDHA), fructose-bisphosphate aldolase A (ALDOA), and SKI oncoprotein (SKI) in the sample, wherein, when compared to a control sample, an increase of the expression level as detected for at least one marker selected from VTNC, BGN, and H10 is indicative for a low risk for recurrence, and a decrease of the expression level as detected for at least one marker selected from LDHA, ALDOA, and SKI is indicative for a high risk for recurrence.

Item 2: The method for determining the risk for recurrence in a bile duct cancer (CCA) patient according to Item 1, wherein the least one marker is selected from the group consisting of VTNC, ALDO, and a combination of all six markers.

Item 3: The method for determining the risk for recurrence in a bile duct cancer (CCA) patient according to Item 1 or 2, wherein the tissue sample is selected from a tumor sample from an intrahepatic (iCCA), perihilar and distal adenocarcinoma, preferably taken at the time of initial surgery.

Item 4: The method for determining the risk for recurrence in a bile duct cancer (CCA) patient according to any one of Items 1 to 3, wherein said method is performed as a first line-therapy testing all six markers to determine the risk of an iCCA patient shortly after initial surgery.

Item 5: The method for determining the risk for recurrence in a bile duct cancer (CCA) patient according to any one of Items 1 to 4, wherein the detection of the expression of the at least one marker in the sample comprises a method selected from at least one of ELISA, immunohistochemical (IHC) staining, proteolysis, bicinchoninic acid assay, and mass spectrometry, preferably comprising a label-free quantitation (LFQ).

Item 6: The method for determining the risk for recurrence in a bile duct cancer (CCA) patient according to any one of Items 1 to 5, wherein the patient is selected from a cat, dog, mouse, rat, horse, sheep, goat, monkey, cow, or human, preferably a human.

Item 7: A method for providing a prognosis for a bile duct cancer (CCA) patient, comprising performing the method according to any one of Items 1 to 6, and further comprising the step of providing a prognosis for the patient based on the risk for recurrence as determined, wherein, when compared to a control sample, an increase of the expression level as detected for at least one marker selected from VTNC, BGN, and H10 is indicative for a positive prognosis, and a decrease of the expression level as detected for at least one marker selected from LDHA, ALDOA, and SKI is indicative for a negative prognosis, wherein optionally the prognosis includes time-to-recurrence (TTR) and/or survival of the patient.

Item 8: A method for monitoring the status of bile duct cancer (CCA) in a patient, comprising performing the method according to any one of Items 1 to 6, and further comprising the step of monitoring the status of bile duct cancer (CCA) in the patient based on comparing the results to results as obtained with an earlier sample obtained from the patient.

Item 9: A method for identifying a bile duct cancer (CCA) patient in need of adjuvant therapy, comprising performing the method according to any one of Items 1 to 6, and further comprising the step of determining the patient as in need of adjuvant therapy, based on the risk for recurrence as determined.

Item 10: The method according to Item 9, wherein the adjuvant therapy is selected from the group consisting of radiation therapy, chemotherapy, immunotherapy, hormone therapy, and targeted therapy.

Item 11: The method according to any one of Items 7 to 10, wherein the patient is undergoing an anti-cancer therapy, in particular a chemotherapy, surgery, radiation, or androgen ablation.

Item 12: A method for identifying a compound that is active against a recurrence in a bile duct cancer (CCA) patient, comprising the steps of: a) providing at least one candidate compound with a subject suffering from CCA, and b) performing the method according to any one of Items 1 to 6, wherein, when compared to the absence of the at least one candidate compound, an increase of the expression level as detected for at least one marker selected from VTNC, BGN, H10, LDHA, ALDOA, and SKI is indicative for a compound that is active against a recurrence.

Item 13: The method according to Item 12, wherein the candidate compound is selected from the group consisting of a chemical molecule, a molecule selected from a library of small organic molecules, a molecule selected from a combinatory library, an anti-cancer chemotherapeutic agent, a cell extract, in particular a plant cell extract, a small molecular drug, a protein, a protein fragment, a molecule selected from a peptide library, and an antibody or fragment thereof.

Item 14: A method for producing a pharmaceutical composition that is active against a recurrence in a bile duct cancer (CCA) patient, comprising the steps of performing the method according to Item 12 or 13, and formulating the at least one compound as identified together with a pharmaceutically acceptable carrier.

Item 15: A method for treating or preventing recurrence in a bile duct cancer (CCA) patient, comprising performing a method according to Item 14, and administering an effective amount of the pharmaceutical composition as produced to the patient, wherein the CCA is preferably selected from the group of intrahepatic (iCCA), perihilar and distal adenocarcinomas.

Item 16: The compound that is active against a recurrence in a bile duct cancer (CCA) patient as identified according to Item 12 or 13, or the pharmaceutical composition as produced according to Item 14 for use in the prevention or treatment of recurrence in a bile duct cancer (CCA) patient.

Item 17: An array of markers for determining the risk for recurrence in a bile duct cancer (CCA) patient, comprising at least one marker selected from Vitronectin (VTNC), biglycan (BGN), histone 10 (H10), L-lactate dehydrogenase A chain (LDHA), fructose-bisphosphate aldolase A (ALDOA), and SKI oncoprotein (SKI).

Item 18: A kit for determining the risk for recurrence in a bile duct cancer (CCA) patient, comprising materials for performing the method according to any one of Items 1 to 13, such as, for example, materials for label-free quantitation (LFQ), buffers, antibodies specifically binding to the markers, and/or the array of markers according to Item 17.

The invention will now be described further in the following examples with reference to the accompanying figures, nevertheless, without being limited thereto. For the purposes of the present invention, all references as cited are incorporated by reference in their entireties.

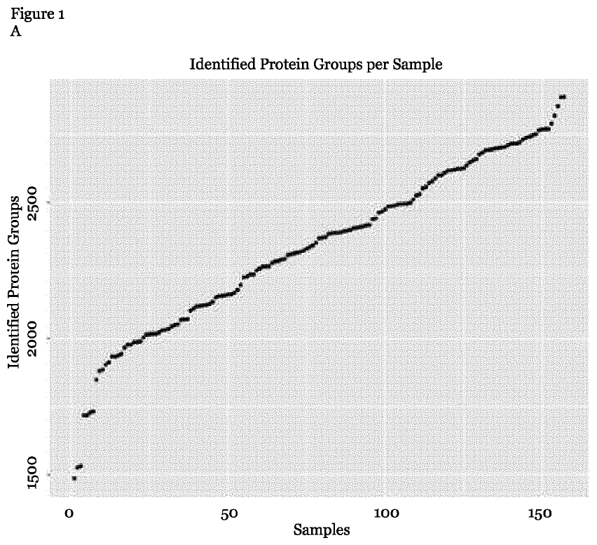

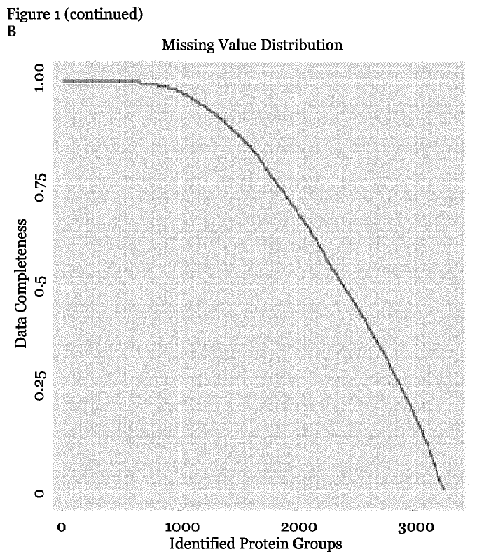

FIG. 1 shows the identified protein groups and data completeness. (A) Numbers of identified protein groups per sample (B) Distribution of missing values across identified protein groups.

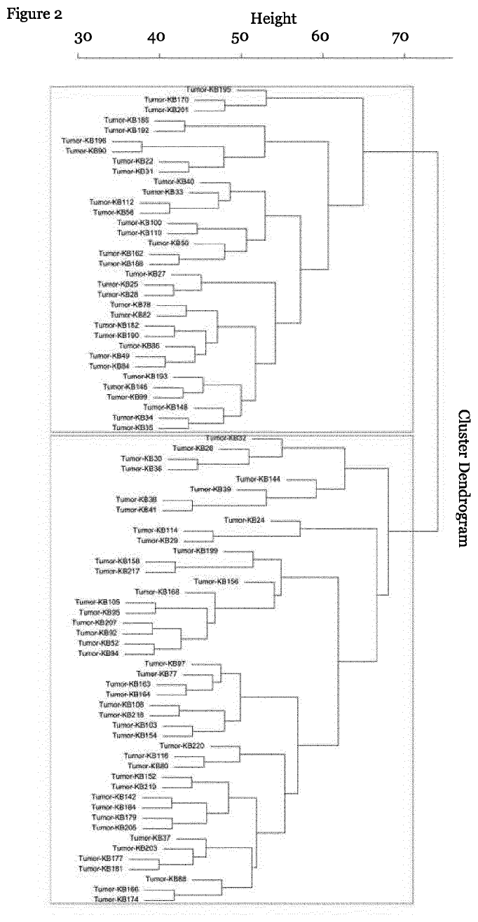

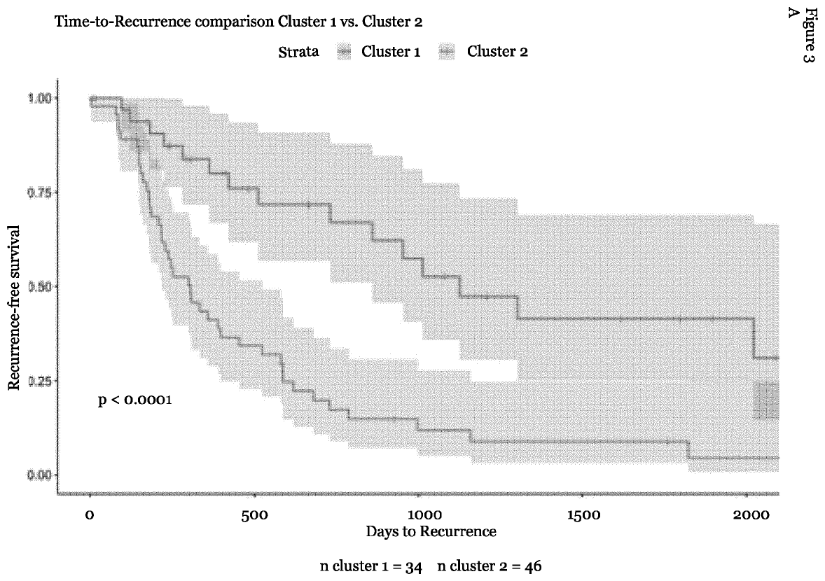

FIG. 2 shows the hierarchical clustering of tumor samples. Cluster dendrogram of hierarchical clustering result with grouping into two subclusters.

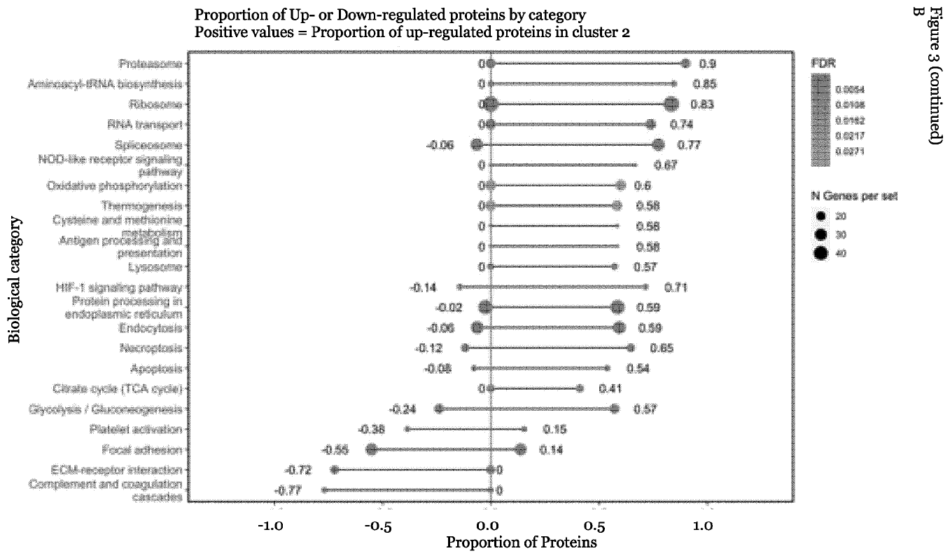

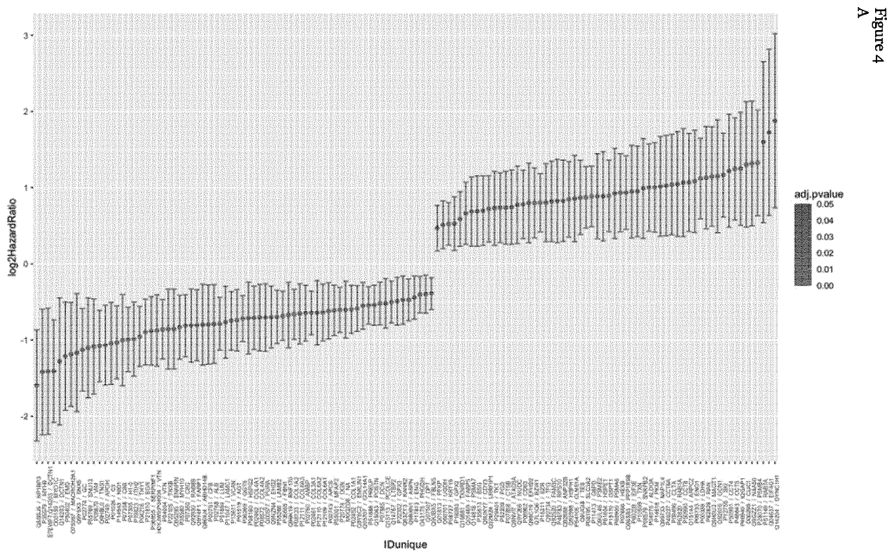

FIG. 3 shows the differences in Survival and Gene Ontology between clusters. (A) Kaplan-Meier plot of TTR prognosis among clusters. (B) KEGG Gene set enrichment comparing cluster 1 with cluster 2.

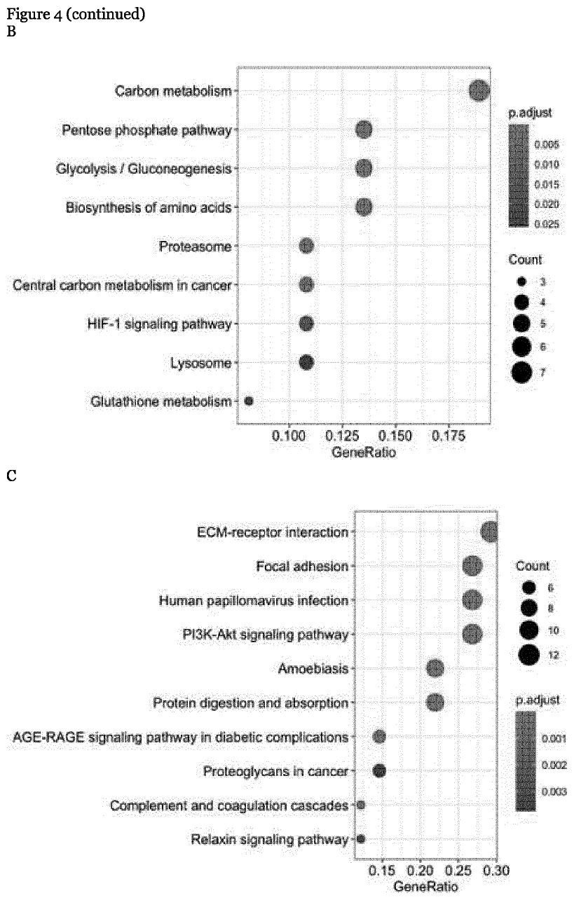

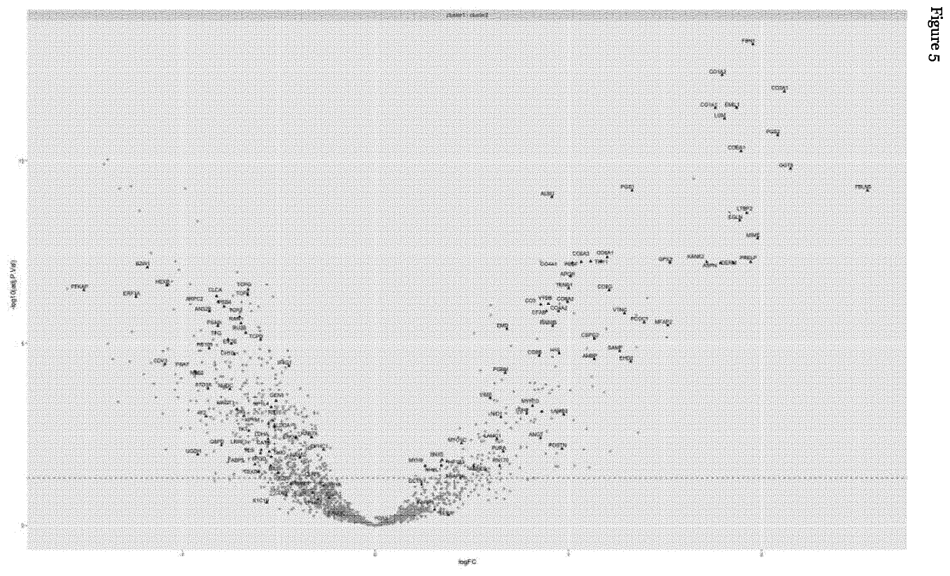

FIG. 4 shows the Cox proportional hazards model results and gene set enrichment. (A) Protein hits and corresponding hazard ratio distribution after CPHM and multiple testing correction. Gene ontology of proteins associated with a high risk (B) and low risk (C) for early recurrence.

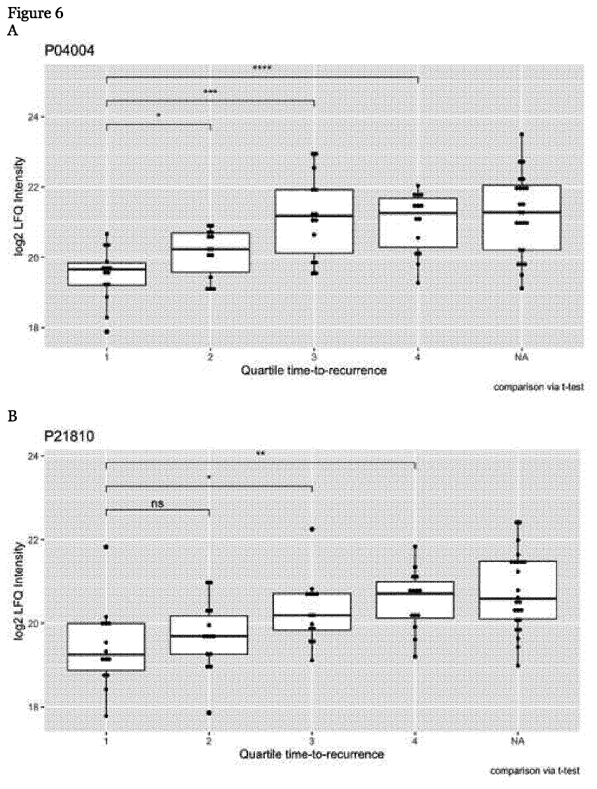

FIG. 5 shows the overlay of results from clustering and Cox′ proportional hazards model. Limma comparison for differentially expressed proteins among cluster 1 and cluster 2. Proteins that were also detected via CPHM are named and highlighted in black.

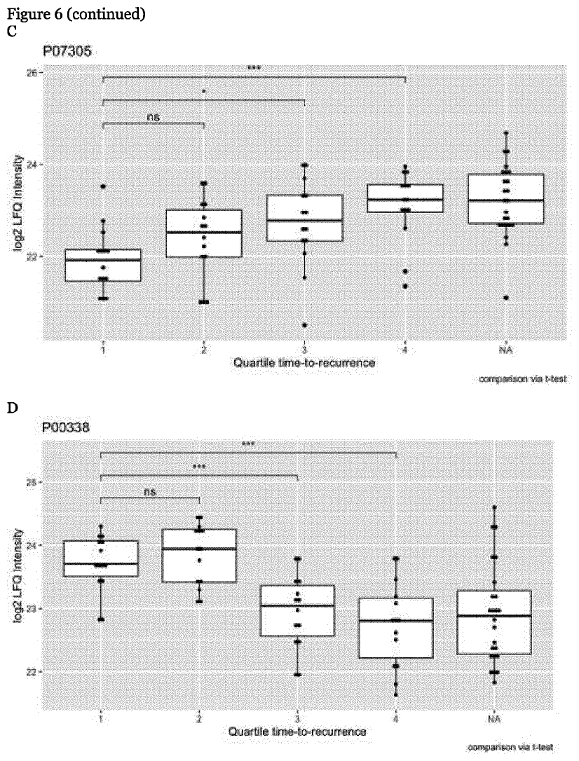

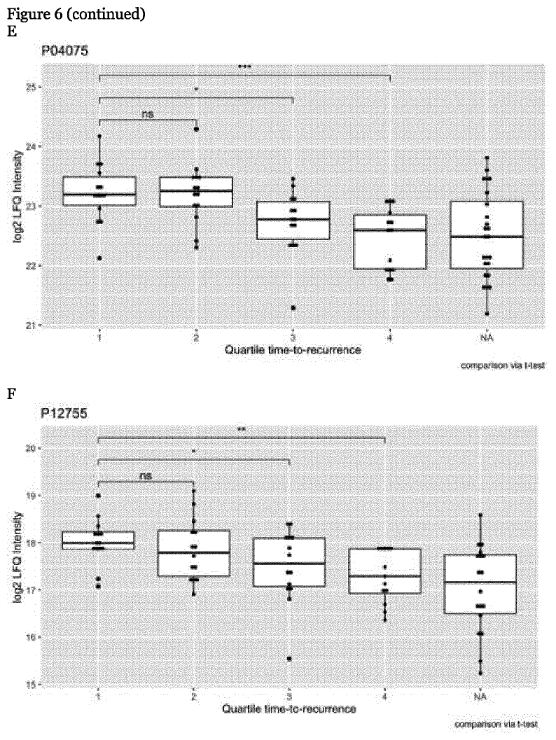

FIG. 6 shows the LFQ intensities of TTR quartiles. The cohort was divided into TTR quartiles to confirm that each marker candidate presents with a sufficient log fold-change in LFQ intensity over the TTR. Censored patients were grouped as ‘NA’.

### EXAMPLES

In the context of the present invention, the inventors have investigated iCCA tissue via liquid-chromatography mass-spectrometry (LC-MS) for tumor resident proteomic profiles that allow predictions about patient's risk for recurrence as reflected by time-to-recurrence (TTR). Since surgery constitutes the first line-therapy tumor tissue is readily available, which allows the circumvention of surrogate markers. Moreover, its collection does not represent an additional burden to the patient.

The cohort consisted of 80 tumor-and 77 matched tumor adjacent non-malignant tissue samples derived from 80 patients. All formalin-fixed, paraffin-embedded (FFPE) samples were macro-dissected and great care was taken to exclude fibrotic or hepatocellular tissue. To prepare samples for LC-MS measurement, the tissue was disintegrated through sonication and boiled at 95 °C. Extracted proteins were reduced with dithiothreitol (DDT), alkylated with iodoacetamide, and digested with lysyl-endopeptidase (LysC) for two hours and with trypsin overnight. Afterwards, samples were acidified with trifluoroacetic acid, desalted via PreOmics solid phase columns and vacuum dried. LC-MS measurements of resuspended samples were conducted in data independent acquisition (DIA) mode with 120-minute gradients. To assemble a library of detectable features, peptides from 24 representative samples were concatenated and measured in 6 gas-phase fractions.

Library generation, Peptide to Spectrum Matching (PSM) and label-free quantification was performed in DIA-NN and all subsequent data analysis was conducted with various R packages. After PSM, on average 2318 protein groups could be identified per sample, 1719 of which showed less than 20% missing values. To remove measurement bias, all data was log2 transformed, normalized and batch corrected via the ComBat algorithm. Since the inventors investigated proteomic profiles associated with patient individual TTRs, all following analyses were performed on the 80 tumor tissue samples only.

### Statistical Analysis

The inventors could group samples according to their similarity via an unsupervised hierarchical clustering algorithm. Subsequent Monte-Carlo simulation suggested that most reliable subgrouping can be achieved by slicing the data into two distinct clusters. Via Kaplan-Meier analysis, the inventors compared patient TTRs between both clusters. With a high degree of statistical confidence, inclusion into cluster 1 signified a beneficial prognosis, while patients grouped into cluster 2 showed faster disease deterioration. Most patients who did not develop a recurrence, and thus were censored from survival statistics, were also grouped into cluster 1. Linear Modeling (Limma) analysis of differentially expressed proteins followed by gene set enrichment via KEGG and REACTOME databases revealed distinct biological processes to be upregulated in either cluster.

In cluster 1, a fingerprint of matrisomal processes, including collagen synthesis, extracellular matrix (ECM) signaling and focal adhesion, was detected. Cluster 2 revealed an increased activity in synthesis and processing of RNA and proteins.

In a second analysis, which was entirely independent of the clustering approach, the inventors iteratively applied Cox′ proportional hazards model (CPHM) to each protein with more than 70% data completeness. Thus, the inventors could identify single proteins whose expression correlates with TTR distribution. The resulting list of protein candidates was adjusted for multiple-testing errors via Benjamini-Hochberg correction. 130 proteins could be found to have expression patterns connected to the TTR. Of these, 60 showed a positive hazard ratio, which indicates a higher risk for early recurrence. Gene set enrichment analysis via KEGG database revealed a large fraction of these proteins to be involved in carbon/glucose metabolism and protein turnover. Conversely, 70 proteins were associated with a low hazard ratio and reduced risk for early recurrence, most of which seemed to be involved in ECM processes like focal adhesion.

Since both statistical approaches uncovered similar biological motifs to be contingent on the TTR, the inventors compared protein hits from both analyses. Differentially expressed proteins between the clusters from approach 1 were visualized in a volcano plot, in which the inventors emphasized all 130 protein hits of the second CPHM approach. Both analyses showed largely overlapping results, indicating that the inventors' protein hits indeed might function as marker candidates to estimate patient's TTRs.

A set of particularly relevant proteins with significant, adjusted p-values was selected according to the following criteria:
Very high or low hazard ratio in CPHM;
Expression shows strong log fold-change over TTR;
Strong differential expression among clusters;
Low adjusted p-value; and
Representative for biological motifs identified as being involved in TTR.

The inventors decided to include into the marker candidate panel 3 proteins associated with low-and 3 proteins with high risk for early recurrence. Each of them showed a strong log fold-change in LFQ intensity over the TTR, which enabled subsequent immunohistochemical (IHC) staining. The selected proteins were:
Vitronectin (VTNC-P04004): cell adhesion and spreading factor;
Biglycan (BGN-P21810): involved in collagen fiber assembly;
Histone H1.0 (H10-P07305): found in cells in terminal stages of differentiation or that have low rates of cell division;
L-lactate dehydrogenase A chain (LDHA-P00338): enzyme involved in pyruvate fermentation;
Fructose-bisphosphate aldolase A (ALDOA-P04075): key enzyme in glycolysis and gluconeogenesis; and
SKI oncoprotein (SKI-P12755): repressor of TGFβ signaling.

Because these protein candidates might be involved in the underlying biology of TTR differences, the inventors investigated them for potential pro- or anti-tumorigenic properties. Since an upregulation of matrisomal processes was linked to a beneficial prognosis and improved survival, the inventors focused on Transforming-Growth-Factor β signaling (TGF-β), which is a master regulator of ECM protein expression with protective effects against CCA.

One high-risk protein candidate, SKI oncoprotein, is known to be a major inhibitor of TGF-β signaling. Therefore, the inventors hypothesize that TGF-β mediated ECM synthesis and its protection against fast tumor recurrence are abrogated by increased SKI expression.

### REFERENCES AS CITED

Banales, J. M. et al. Cholangiocarcinoma 2020: the next horizon in mechanisms and management. Nat. Rev. Gastroenterol. Hepatol. 17, 557-588 (2020).
Kelley, R. K., Bridgewater, J., Gores, G. J. & Zhu, A. X. Systemic therapies for intrahepatic cholangiocarcinoma. J. Hepatol. 72, 353-363 (2020).
Gillet, L. C., Leitner, A. & Aebersold, R. Mass Spectrometry Applied to Bottom-Up Proteomics: Entering the High-Throughput Era for Hypothesis Testing. Annu. Rev. Anal. Chem. 9, 449-472 (2016).
Tecalco-Cruz, A. C., Ríos-López, D. G., Vázquez-Victorio, G., Rosales-Alvarez, R. E. & Macías-Silva, M. Transcriptional cofactors Ski and SnoN are major regulators of the TGF-B/Smad signaling pathway in health and disease. Signal Transduct. Target. Ther. 3, 1-15 (2018).
Mu, X. et al. Epithelial Transforming Growth Factor-β Signaling Does Not Contribute to Liver Fibrosis but Protects Mice from Cholangiocarcinoma. Gastroenterology 150, 720-733 (2016).

1-16. (canceled)

17. A method for determining the risk for recurrence in a bile duct cancer (CCA) patient, comprising:
detecting an expression level of at least the marker Vitronectin (VTNC) in a tissue sample obtained from the patient,
wherein, when compared to a control sample, an increase of the expression level as detected for the marker VTNC is indicative for a low risk for recurrence, and
wherein the patient is selected from a cat, dog, mouse, rat, horse, sheep, goat, monkey, cow, or human.

18. The method for determining the risk for recurrence in a bile duct cancer (CCA) patient according to claim 17, wherein, in addition to the expression level of VTNC, expression levels of markers biglycan (BGN), histone 10 (H10), L-lactate dehydrogenase A chain (LDHA), fructose-bisphosphate aldolase A (ALDOA), and SKI oncoprotein (SKI) are also detected.

19. The method for determining the risk for recurrence in a bile duct cancer (CCA) patient according to claim 17, wherein the tissue sample is selected from a tumor sample from an intrahepatic (iCCA), perihilar and distal adenocarcinoma.

20. The method for determining the risk for recurrence in a bile duct cancer (CCA) patient according to claim 18, wherein said method is performed as a first line-therapy testing all six of said markers to determine the risk of an iCCA patient shortly after initial surgery.

21. The method for determining the risk for recurrence in a bile duct cancer (CCA) patient according to claim 17, wherein the detection of the expression of the at least one marker in the sample comprises a method selected from at least one of ELISA, immunohistochemical (IHC)

22. A method for providing a prognosis for a bile duct cancer (CCA) patient, comprising performing the method according to claim 18, and further comprising the step of providing a prognosis for the patient based on the risk for recurrence as determined, wherein, when compared to a control sample, an increase of the expression level as detected for a marker selected from VTNC, BGN, and H10 is indicative for a positive prognosis, and a decrease of the expression level as detected for a marker selected from LDHA, ALDOA, and SKI is indicative for a negative prognosis.

23. A method for monitoring the status of bile duct cancer (CCA) in a patient, comprising performing the method according to claim 17, and further comprising the step of monitoring the status of bile duct cancer (CCA) in the patient based on comparing the results to results as obtained with an earlier sample obtained from the patient.

24. A method for identifying a bile duct cancer (CCA) patient in need of adjuvant therapy, comprising performing the method according to claim 17, and further comprising the step of determining the patient as in need of adjuvant therapy, based on the risk for recurrence as determined.

25. The method according to claim 24, wherein the adjuvant therapy is selected from the group consisting of radiation therapy, chemotherapy, immunotherapy, hormone therapy, and targeted therapy.

26. The method according to claim 22, wherein the patient is undergoing an anti-cancer therapy.

27. A method for identifying a compound that is active against a recurrence in a bile duct cancer (CCA) patient, comprising the steps of: a) providing at least one candidate compound with a subject suffering from CCA, and b) performing the method according to 17, wherein, when compared to the absence of the at least one candidate compound, an increase of expression level as detected for the marker VTNC is indicative for a compound that is active against a

28. A method for producing a pharmaceutical composition that is active against a recurrence in a bile duct cancer (CCA) patient, wherein the method comprises the steps of performing the method according to claim 27, and formulating the at least one compound as identified together with a pharmaceutically acceptable carrier.

29. A kit for determining the risk for recurrence in a bile duct cancer (CCA) patient, wherein the kit comprises materials for label-free quantitation (LFQ), buffers, antibodies specifically binding to marker VTNC, and/or binding to the markers Vitronectin (VTNC), biglycan (BGN), histone 10 (H10), L-lactate dehydrogenase A chain (LDHA), fructose-bisphosphate aldolase A (ALDOA), and SKI oncoprotein (SKI).
